SEQUENCE LISTING (4 sequences)

SEQ ID NO: 1 - protein, 1269 aa, synthetic construct
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLDVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGVCASYQTQTKSRQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3810 nt, synthetic construct
  source 1..3810: mol_type = other RNA
atgtttgtgtttttggtcctgctgccattggtctcctctcagtgcgtgatgccactcttcaacctcataaccacaacacaaagttatactaatagttttactcgaggagtgtattaccccgataaagtcttcagatcttccgtccttcatctgacgcaggatctcttcttgccctttttctccaacgttacctggtttcacgcgattagtggcaccaacggaactaaaagatttgataatcccgttcttccgttcaatgatggcgtctattttgccagcacagaaaaaagtaacatcattagaggatggatctttggcacgacgctggattccaaaacccaaagccttctcatagttaataatgctacaaatgtgtttataaaagtttgtgaatttcaattctgtaatgatccgttcctcgacgtatatcataagaataataagtcctggatggagtcagagagtggtgtatatagttcagccaataactgcacttttgagtacgtatcccaacctttcctcatggatctcgaagggaagcaggggaattttaaaaatcttagagagtttgtttttaagaatatcgacgggtatttcaaaatttattccaaacataccccaattataggccgggacttccctcaggggttctctgcgcttgaacccttggtggatctgcccataggcatcaacataacacgctttcagacccttttggctctgaacaggtcctacctcacaccgggcgatagctcctcaggatggaccgccggcgccgcggattattacgttggttatcttcaacctagaacattccttctcaaatacaatgaaaatggaactattactgacgccgtagactgcgctcttgaccccttgtcagagacaaagtgtacccttaagtcattcaccgtcgaaaagggaatttaccagacttctaatttccgggtccagccaaccgagagtatcgttcgattcccgaacgtaacaaacctgtgcccgttccatgaggtctttaacgctactcggttcgcttcagtttatgcatggaaccgaaccagaatttctaactgtgtcgccgattactctgtgctgtacaactttgcgcctttttttgccttcaaatgttacggcgtgagcccaaccaaactgaacgacctttgctttactaacgtctacgcggacagcttcgtcatcaagggaaacgaagtgtcccagatagcgcctgggcagacaggaaacattgcggattacaattacaagctgccggacgacttcacgggatgtgttattgcgtggaattcaaataagctcgattccaagcatagtggtaattatgattattggtacaggagttttagaaagtctaagttgaagcccttcgaaagagatatcagcactgaaatttatcaagctgggaacaaaccctgcaaaggtaagggaccgaactgttactttcctcttcaaagctacgggttccgcccgacttatggcgtaggacaccagccttaccgcgtagtggtgctgagtttcgagttgcttcatgcaccagcaacagtctgtggtccgaagaagagcaccaacttggtcaagaacaagtgcgtaaacttcaattttaatgggctgactggtacgggagtactcacgaagagcaacaaaaagtttttgccatttcaacaatttggccgcgatatagtcgatacaaccgatgccgtacgggatccgcaaaccctggaaatcctggatatcaccccttgttcctttggtggagtttctgttattaccccaggaacgaacacatccaatcaggtcgctgtgctttatcagggtgtcaactgcacggaggttagtgttgcaatacatgccgatcaactgacacccacctggcgggtgtacagtaccgggtctaatgtatttcagacccgggctggttgtctgattggtgcggagtatgtaaacaactcttacgagtgcgacataccgattggcgccggggtgtgtgcctcctatcaaacccaaacaaaatctcgccaacaggcccagtccgtcgctagtcagagcattattgcttacactatgagtctgggggctgagaactcagtcgcttatagtaataacagcatagcgatacctacaaacttcactatttccgttaccacagagatactcccggtttccatgactaagacgagcgtcgattgcactatgtatatctgtggggacagcactgaatgtagcaacttgctcttgcagtatggctcattttgtacgcaactcaagcgcgctcttacgggtatcgcggtagagcaagacaaaaatactcaggaagtgtttgcgcaggtaaaacagatttataagaccccgccaatcaagtattttgggggttttaacttttcacaaatcttgcccgacccatctaagccatcaaaaaggagcttcatagaagatcttctcttcaataaagtgaccctcgcggacgccggctttatcaaacagtacggtgactgccttggtgatatcgccgctagagatcttatctgtgcgcagaagtttaatggactgaccgtgctcccgccactcctcaccgatgagatgatagcccagtatacaagtgccctcctcgcaggaacgattacttcagggtggacgtttggagcgggcgcggcgctgcaaataccctttgcgatgcagatggcatatcgattcaacgggatcggtgtcacccaaaatgtcctgtacgaaaatcagaagctcatcgctaatcagtttaattcagccataggtaaaatacaagactcattgttttccacggcttctgctctcggtaagctgcaagatgttgtgaatcataatgcccaagctctgaatacgttggtaaagcagttgtcaagtaaatttggagcgatctccagcgtacttaatgacatactttcccgactggatccaccggaagcggaagttcagattgatcggctcattacaggacgcctccagtctcttcagacttacgttacacaacagctgatcagagccgctgaaatacgggcaagtgcgaatcttgccgcgacaaagatgtcagagtgtgtgctcgggcagagtaagcgagtagatttctgcggcaaagggtaccaccttatgtcatttccgcaatcagcacctcatggggtcgtctttcttcatgttacatatgtaccagcacaggaaaaaaattttactacagcgcccgcaatatgccacgatggcaaggcgcactttccccgagagggagtctttgtatccaatggaacccattggtttgtgactcagagaaacttttacgagccacagatcataaccacagacaatacatttgtttccggaaattgcgacgtcgtgatcggaattgtcaataatacagtctacgatcccctccaactcgaactggacagtttcaaagaggagctggacaagtatttcaagaaccacactagtccagatgttgatttgggcgacatctctggtatcaacgcaagtgtagtcaatattcaaaaggaaatagatcgactcaatgaggtcgcaaagaatcttaacgagtcactgattgatctccaggagctgggcaagtacgaacaatacataaaatggccctggtatatctggcttggtttcatagcgggcttgatcgcgattgtaatggtcacgataatgctttgttgtatgacttcttgttgtagttgcttgaaagggtgttgttcatgcggtagctgttgtaaattcgacgaagatgattcagaacctgtacttaaaggtgtgaagctgcactacacatga

SEQ ID NO: 3 - protein, 1269 aa, synthetic construct
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLDVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATTFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSLRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFLTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - RNA, 3810 nt, synthetic construct
  source 1..3810: mol_type = other RNA
atgttcgtatttttggtacttctgcctctcgtcagctcccagtgcgtcatgccactctttaacttgatcactactactcaatcctacacgaactctttcacccggggtgtatactatccagacaaggtatttcggagctctgtattgcatcttactcaagacttgttccttcctttcttctccaacgtgacatggtttcatgccatatccgggacaaatggtacaaagcgatttgacaaccccgttcttccattcaatgatggcgtctactttgcaagtactgagaaatccaacattattaggggatggatcttcggaactacgttggattctaagacccaaagtttgttgattgtcaacaacgcaactaacgtctttattaaggtttgtgaatttcagttttgcaatgatccttttcttgacgtataccataaaaataacaagtcatggatggaaagcgagtccggtgtatatagtagcgcgaacaactgcacgtttgagtatgtaagccagccttttctcatggacctggaagggaaacagggaaatttcaaaaatctccgagagttcgtatttaagaacatagacgggtacttcaaaatttatagcaaacatacgcccattattggtcgcgacttcccccaaggatttagcgcacttgagcccctggttgacctgccgattggaataaatataactcgattccagacactgctcgcacttaatcggtcttatttgacgcctggggattcctctagtggatggaccgcaggggccgctgattattacgtcggctacttgcagccgaggacctttttgcttaaatataatgaaaacggtacaattactgatgcagtcgattgcgctctggatccgcttagcgaaaccaaatgcactctcaaatctttcacagtcgaaaaaggtatttatcagacctctaacttccgggtgcagccgactgagtccatagttcggttcccaaatgtgacgaatttgtgcccatttcatgaggtgttcaatgcaacgacgttcgcatcagtctatgcctggaaccggacccgaatctctaattgtgtagccgactacagtgttctgtataattttgctcccttctttgcgttcaaatgctacggggtgagtcccaccaaacttaatgacttgtgcttcacaaacgtctacgctgattcattcgtcatcaaaggtaacgaggtaagtcagatagcgccgggccagaccgggaacatcgcggactacaattataagttgccagatgacttcactgggtgtgtcatcgcgtggaacagcaataaacttgattctaaacattccggaaactatgattactggtataggagtttgcggaaaagcaaactcaaaccgtttgagagggatatatccacggagatatatcaagcaggcaacaagccatgcaaggggaagggacctaactgctacttccctcttcaaagttatgggttcagaccgacatacggggtcggccaccagccttatcgcgttgtagtcctcagctttgaattgttgcacgctccggccactgtttgtggcccgaagaaatccactaacttggttaagaataaatgtgtaaacttcaatttcaacgggctgacagggaccggtgtgctcacgaagtcaaacaagaaattcctgcctttccaacagttcgggcgcgatatagtggataccactgacgctgtgcgagacccccaaactctggaaatactggacatcaccccgtgctcttttggaggtgttagtgtaatcacaccgggaacgaatacgagcaatcaagttgccgttctctaccaaggagtcaactgcacggaggtatctgttgcaatacatgcggatcaattgactcccacgtggagggtttattctacgggttccaatgttttccagacgcgggccggctgtctcattggcgcggaatacgttaacaactcctacgagtgtgatattcctattggagcaggtatttgtgctagctatcagacgcaaaccaaaagccgacaacaggcccagagcgtagccagtcaatcaattatagcatacactatgtcactgggcgccgagaacagtgttgcctatagcaacaacagcatagccatccctacaaattttacaatctcagtgactacggaaattttgcctgtctcaatgacgaagactagcgttgactgcactatgtacatatgtggggattcaaccgaatgcagtaatcttctccttcaatatgggtccttttgtactcaattgaaaagggccttgacaggcatagccgttgagcaggacaaaaatacccaggaggtcttcgcccaagtaaagcaaatttacaagacgccccccataaagtatttcggtggctttaacttttctcaaatactgccggatccatctaagcctagcaaacgctctttcatagaagacttgctcttcaacaaagtaacactcgccgatgcgggtttcattaagcagtacggagattgtctcggggatattgcagctagagacctgatttgcgcccaaaagtttaatgggttgacggtattgcctccactgttgactgacgagatgatcgcccagtacacctcagcgttgttggctggaacgatcacatctggatggacgtttggggctggtgcggcacttcagataccctttgcgatgcaaatggcgtatcggttcaatgggatcggcgtgacgcagaacgtgttgtacgagaatcaaaaactcatagccaaccagtttaacagtgcgataggtaaaattcaggactccttgttctccaccgcctcagcactcggtaaactgcaagatgtagtgaatcacaacgctcaagcattgaacactttggtaaagcagctctcctcaaagtttggagctatctcctcagttcttaatgatatcctctctaggctggatccgccggaggccgaagtgcaaatcgatcgactcattacagggcgattgcagagccttcagacatatgttactcaacaacttataagggcggccgagattcgggcttccgccaatctggcagcaacaaagatgtccgagtgcgtccttgggcagtccaagcgcgtcgatttctgtggaaagggatatcatcttatgagttttccgcagtcagcaccacatggagttgtgtttctccacgttacctatgtaccagcacaagagaaaaacttcaccacagccccggctatctgtcatgatggaaaggcacacttcccccgcgagggcgtgttcgtgagtaacgggacgcattggttcctgacgcaaagaaatttttatgagcctcagataataactaccgataatacttttgtctcaggaaactgcgatgttgtaatcggcatcgtcaataataccgtctatgatccactgcagctcgaacttgactccttcaaggaagagctcgacaagtacttcaagaatcatactagtcctgatgttgacctgggcgacatctctggcattaatgcctctgtcgtaaacattcaaaaggaaatcgatagacttaatgaggtcgctaagaacttgaacgaatctctgatagaccttcaggaactgggtaaatatgagcagtatataaagtggccttggtacatctggcttgggttcattgcgggtttgatcgcgatagtaatggtaacaatcatgttgtgttgcatgacatcctgctgttcctgcctcaagggctgctgttcttgcggatcctgctgtaagttcgatgaggatgattcagagccggttttgaagggagttaagctgcattatacttga

(19) United States
(12) Patent Application Publication
(10) Pub. No.: US 2026/0199452 A1
(43) Pub. Date: Jul. 16, 2026

(54) RIBONUCLEIC ACID COMPOSITIONS HAVING ENHANCED IMMUNOGENICITY

(71) Applicant: NOVAVAX, INC., Gaithersburg, MD (US)

(72) Inventors: Gale SMITH, Gaithersburg, MD (US); Nita PATEL, Gaithersburg, MD (US)

(21) Appl. No.: 19/446,685

(22) Filed: Jan. 12, 2026

Related U.S. Application Data
(60) Provisional application No. 63/744,020, filed on Jan. 10, 2025.

(51) Int. Cl. A61K 39/215; A61K 39/00; A61K 39/145; A61P 37/04

(52) U.S. Cl. CPC A61K 39/215; A61K 39/145; A61P 37/04; A61K 2039/53; A61K 2039/55555; A61K 2039/55577; A61K 2039/70

(57) ABSTRACT

Disclosed herein are immunogenic compositions comprising ribonucleic acids (RNA) and combination vaccines that include saponin-based adjuvants, as well as methods of using the immunogenic compositions. Methods for preparing the immunogenic compositions, and methods of administering the immunogenic compositions are also disclosed.

### RELATED APPLICATION

The present application claims priority to U.S. Provisional Patent Application No. 63/744,020, filed on Jan. 10, 2025, the contents of which is incorporated by reference in its entirety.

### REFERENCE TO AN ELECTRONIC SEQUENCE LISTING

This application contains a Sequence Listing which has been submitted electronically in XML format and is hereby incorporated by reference in its entirety. Said XML copy, created on Jan. 12, 2026 is named 1450_123US1_Sequence_Listing_01_12_2026 and is 14,739 bytes in size.

### FIELD OF THE INVENTION

The present invention generally relates to immunogenic compositions comprising ribonucleic acids (RNA) and combination vaccines, as well as methods of using the immunogenic compositions. The immunogenic compositions of the present invention comprises a saponin based adjuvant. The invention also provides methods for producing the immunogenic compositions and methods of stimulating immune responses with the compositions. The invention also provides methods of administering the immunogenic compositions.

### BACKGROUND OF THE INVENTION

Influenza and coronavirus disease 2019 (COVID-19) are life-threatening illnesses caused by the viruses influenza virus and sudden acute respiratory coronavirus 2 (SARS-CoV-2), respectively. The fatality rate of patients diagnosed with influenza is approximately 0.1%, and the case fatality rate of patients diagnosed with COVID-19 ranges from 0.2% to 7.7%.

The development of vaccines that prevent or reduce the severity of these life-threatening infectious diseases is desirable. However, human vaccine development remains challenging because of the highly sophisticated evasion mechanisms of pathogens and difficulties stabilizing vaccines. Optimally, a vaccine must both induce antibodies that block or neutralize infectious agents and remain stable in various environments, including environments that do not enable refrigeration. Combination of two antigens from two pathogens in a single vaccine composition is particularly challenging because the antigens may interact with each other, preventing a sufficient immune response to either pathogen.

Many cancers arise due to mutations, for example point mutations, in a protein, forming neoantigens that drive tumor growth. Immunogenic treatments of different types of cancers are being developed, which do not necessarily target the specific mutation(s) that cause the cancer, and so such treatments may attack a broader spectrum of proteins than is required to attack the cancer. It would be preferred to direct the immunogenic response to specific mutations known to be oncogenic and presented as an oncogenic peptide.

Saponins can be used as adjuvants for killed vaccine antigens e.g. in a multi component vaccine without causing negative effects on the live replicating vaccine components. This is contrary to most (other) commonly used adjuvants that decrease the capacity of the live microorganisms to replicate.

Matrix-M adjuvant is a saponin based adjuvant that enhances acquired B-cell immunity by broadening epitope recognition, increasing neutralizing antibody titers, and inducing long-lasting memory B cells in the plasma and bone marrow. Matrix-M adjuvant also induces early activation of innate cells at the injection site and draining lymph nodes, improving magnitude and quality of acquired responses (Stertman et al., The Matrix-M adjuvant: A critical component of vaccines for the 21^{st} century. 2023. Hum Vacc Imm 19(1)). Matrix-M is known to be taken up by antigen presenting cells (APCs) where the low pH of the lysosome releases saponins, inducing lysosomal membrane destabilization and endocytosis of antigens into the cytosol. Matrix-M adjuvant offers potential for pre-pandemic priming with seasonal vaccines and for enhancing the immunogenic response in cancer treatments.

### SUMMARY OF THE INVENTION

An embodiment of the invention is directed to immunogenic composition, comprising a messenger ribonucleic acid (mRNA) that encodes an antigenic polypeptide, and a saponin-based matrix adjuvant.

In some aspects, the saponin-based matrix adjuvant comprises Matrix-A and Matrix-C.

In some aspects, the Matrix-A and Matrix-C are respectively present at a weight-to-weight ratio of about 85:15.

In some aspects, the saponin-based matrix adjuvant is present in a dose of about 5 μg to about 200 μg.

In some aspects, the composition further comprises a lipid nanoparticle.

In some aspects, the lipid nanoparticle comprises a PEG-modified lipid, a non-cationic lipid, a sterol, an ionizable cationic lipid, or any combination thereof.

In some aspects, the lipid nanoparticle comprises about 0.5-15 mol % PEG-modified lipid; about 5-25 mol % non-cationic lipid; about 25-55 mol % sterol; and about 20-60 mol % ionizable cationic lipid.

In some aspects, the antigenic polypeptide is one of a SARS-CoV-2 spike (S) protein, MERS, SARS, and an influenza virus antigenic polypeptide.

In some aspects, the antigenic polypeptide is a picornavirus capsid polyprotein, wherein the capsid polyprotein comprises a viral P1 precursor polyprotein.

In some aspects, the composition further comprises a recombinant protein antigen.

In some aspects, the recombinant protein antigen comprises hemagglutinin of at least one strain of seasonal influenza.

In some aspects, the recombinant protein antigen comprises hemagglutinin of at least three strains of seasonal influenza.

In some aspects, a dose of the composition produces an immunogenic response, measured using an IgG titer, in a murine subject that is at least twice the immunogenic response generated in a comparable murine subject exposed to the same dose of mRNA, but without the saponin-based adjuvant.

In some aspects, the invention includes method of generating an immune response in a subject by administering the immunogenic composition to the subject.

In some aspects, the method includes combining the mRNA with the saponin-based adjuvant more than 12 hours prior to administering the composition to the subject.

In some aspects, the method includes combining the mRNA with the saponin-based adjuvant more than 24 hours prior to administering the composition to the subject.

In some aspects, the immunogenic composition is used to generate an immune response in a subject.

In some aspects, the immunogenic composition is used for the preparation of a medicament for generating an immune response in a subject.

In some aspects, the preparation comprises combining the mRNA with the saponin-based adjuvant more than 12 hours prior to administering the composition to the subject.

In some aspects, the preparation comprises combining the mRNA with the saponin-based adjuvant more than 24 hours prior to administering the composition to the subject.

Another embodiment of the invention comprises a prefilled syringe comprising the immunogenic composition.

Another embodiment of the invention comprises a kit comprising the immunogenic composition, wherein a first vial contains the mRNA and a second vial contains the saponin-based adjuvant.

In some aspects, the first vial includes space to receive contents of the second vial or the second vial includes space to receive contents of the first vial.

### BRIEF DESCRIPTION OF THE FIGURES

FIG. 1 shows the timeline of the mouse study performed in Example 1.

FIGS. 2A-2H show IgG titer and pseudovirus assay results. FIGS. 2A and 2B respectively present the IgG titer and pseudovirus neutralization titer results for the 5 μg single vaccine doses (Groups 1, 4, and 7). FIGS. 2C and 2D respectively show the IgG titer and pseudovirus neutralization titer results for the 1 μg doses (Groups 2, 5, and 8). FIGS. 2E and 2F illustrate the cross-immunization capability of the 1 μg dose. FIG. 2E shows the results of an anti-KP.3.1.1.rS IgG titer and FIG. 2F shows the results of a pseudovirus neutralization. FIGS. 2G and 2H respectively show the IgG titer and pseudovirus neutralization results for the 0.2 μg doses (Groups 3, 6, and 9).

FIGS. 3A-3C further illustrate cross-immunization. Three bars are shown for each Group corresponding, in order from left to right, to i) anti-KP.2 rS IgG, ii) anti-KP.3.1.1 IgG, and iii) anti-XEC rS IgG. FIG. 3A shows the results of anti-rS IgG titers for the 5 μg dose (Groups 1, 4, and 7). FIG. 3B shows the results of anti-rS IgG titers for the 1 μg dose (Groups 2, 5, and 8). FIG. 3C shows the results of anti-rS IgG titers for the 0.2 μg dose (Groups 3, 6, and 9).

FIGS. 4A-4C present results comparing the performance of the 1 μg doses (Groups 2 and 5) with the doses that included the tetravalent seasonal influenza vaccine (Groups 10 and 11), along with the placebo. FIG. 4A presents anti-KP.2 rS IgG titer results for Groups 2, 5, 10, 11, and placebo. FIG. 4B shows results of different anti-rS IgG titers. Three bars are shown for each Group corresponding, in order from left to right, to i) anti-KP.2 rS IgG, ii) anti-KP.3.1.1 IgG, and iii) anti-XEC rS IgG. FIG. 4C shows the results of hemagglutinin influenza (HAI) titers for Groups 10 (NVAX KP.2+tNIV+Matrix-M), 11 (mRNA+tNIV+Matrix-M), and 12 (placebo). Three bars are shown for each Group corresponding, in order from left to right, to i) A/Wis/22 (H1N1), ii) A/Mass/22 (H3N2), and iii) B/Aus/21 (B/Victoria) influenza strains.

FIGS. 5A and 5B show the results of pseudovirus neutralization assays for Groups 2, 5, 10, 11, and 12. FIG. 5A shows KP.2 pseudovirus neutralization results. FIG. 5B illustrates the results of KP.3.1.1 pseudovirus neutralization.

FIGS. 6A-6C illustrate T cell responses from spleen tissue for Groups 2, 5, and 8, using an intracellular cytokine staining (ICCS assay). FIG. 6A shows the triple Th1 CD4+ T cell response for Groups 2, 5, and 8. FIG. 6B shows cytokine count for four different cytokines, IFN-γ, IL2, TNF-α, and IL4, with stimulation by four SARS-CoV-2 strains, for Group 5 (mRNA+Matrix-M). FIG. 6C shows cytokine count for four different cytokines, IFN-γ, IL2, TNF-α, and IL4, with stimulation by the same four SARS-CoV-2 strains, for Group 8 (mRNA).

FIG. 7A shows the multifunctional CD8+ T cell triple Th1 response for groups 2, 5, and 8. FIG. 7B shows the follicular B helper T (TFH) cell response for Groups 2, 5, 9, and 12. FIG. 7C shows the numbers of germinal center B cells (GC B cells) for Groups 2, 5, 9, and 12.

FIG. 8 shows the timeline of the mouse study performed in Example 2.

FIG. 9A shows homologous (KP.2) pseudovirus neutralization results for Groups 1, 4, 6, 9, and 11 in Example 2. FIG. 9B shows homologous (KP.2) pseudovirus neutralization results for Groups 2 and 7. FIG. 9C shows homologous (KP.2) pseudovirus neutralization results for Groups 3, 5, 8, 10, and 12. FIG. 9D shows heterologous (LP.8.1) pseudovirus neutralization results for Groups 1, 4, 6, 9, and 11. FIG. 9E shows heterologous (LP.8.1) pseudovirus neutralization results for Groups 2 and 7. FIG. 9F shows heterologous (LP.8.1) pseudovirus neutralization results for Groups 3, 5, 8, 10, and 12.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

### DETAILED DESCRIPTION OF THE INVENTION

### Definitions

As used herein, the term “adjuvant” refers to a compound or substance that, when used in combination with an immunogen, augments or otherwise alters or modifies the immune response induced against the immunogen. Modification of the immune response may include magnifying the response or broadening the specificity of either or both antibody and cellular immune responses.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value−10%) to 220 (upper value+10%).

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque, or a laboratory animal such as a mouse, rat, or rabbit. In certain aspects, the subject may be a pet, such as a dog or cat.

As used herein, the term “pharmaceutically acceptable” means being approved, or able to be approved, by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

mRNA Vaccines

An mRNA vaccine is one that uses a copy of mRNA that is used by the subject's body in the synthesis of an antigen protein that would normally be produced by a pathogen, such as a virus. These antigen proteins produce an adaptive immune response. The mRNA may be administered “naked,” but may preferably be accompanied by a vector that provides protection from degradation by ribonucleases and assist in penetration into cells. The vector may include a cationic polymer or a lipid nanoparticle.

The present disclosure provides, in some embodiments, immunogenic compositions that comprise RNA (e.g., mRNA) polynucleotides encoding polypeptides that may be derived from coronaviruses, including but not limited to SARS-CoV-2, for example from SARS-CoV-2, from MERS CoV, and from SARS CoV. mRNA vaccines are commercially available, gaining widespread use to stem the COVID-19 pandemic that started in 2020. Examples of such vaccines include Spikevax™ and MNexspike™, available from Moderna, Cambridge MA and Cominarty™, available from Pfizer-BioNTech, Mainz, Germany.

The present disclosure also provides, in some embodiments, combination immunogenic compositions that comprise at least one RNA (e.g., mRNA) polynucleotide. Also provided herein are methods of administering the RNA (e.g., mRNA) immunogenic compositions, methods of producing the RNA (e.g., mRNA) immunogenic compositions, compositions (e.g., pharmaceutical compositions) comprising the RNA (e.g., mRNA) immunogenic compositions, and nucleic acids (e.g., DNA) encoding the RNA (e.g., mRNA) immunogenic compositions.

In embodiments, the lipid nanoparticle comprises PEG-modified lipid, a non-cationic lipid, a sterol, an ionizable cationic lipid, or any combination thereof. In some embodiments the lipid nanoparticle comprises about 0.5-15 mol % PEG-modified lipid; about 5-25 mol % non-cationic lipid; about 25-55 mol % sterol; and about 20-60 mol % ionizable cationic lipid.

### Recombinant Protein Vaccines

Recombinant protein vaccines are based on proteins formed using recombinant DNA technology. They are isolated protein antigens, unlike conventional protein antigens that use either a killed virus or weakened virus. Use of a recombinant protein avoids the introduction of unnecessary biological components to the body, ensuring that the subject's immune system training is focused on the specific purified recombinant antigen used in the vaccine. Recombinant protein vaccines have been available since the 1980s, with the first recombinant hepatitis B vaccine. Other examples include recombinant hemagglutinin (HA) used for vaccination against treatment seasonal influenza. Recombinant proteins have also been used in vaccines against other infections such as respiratory syncytial virus (RSV).

### Adjuvants

Adjuvants containing saponin can also be combined with the chimeric proteins disclosed herein. Saponin-based adjuvants, i.e. adjuvants containing saponin, may also be used. Saponins are glycosides extracted from the bark of the Quillaja saponaria Molina tree. These bark extracts contain a heterogeneous mixture of hundreds of related saponins with structurally different glycosylation or acylation patterns that also affect their biological activities.

In some embodiments, the compositions disclosed herein may be combined with one or more adjuvants to enhance an immune response. In other embodiments, the compositions are prepared without adjuvants and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

Saponins are a large family of plant-derived glycoconjugates that share a triterpene structure with a variety of glycoside side chains. Saponins have traditionally been used for making soaps based on being amphipathic. Saponins now also are used for making adjuvants based on having potent immune-stimulating properties, as taught, for example by Kensil et al., U.S. Pat. No. 5,057,540.

Saponins extracted from the bark of the South American soapbark tree Quillaja saponaria Molina contain a complex heterogeneous mixture of closely related saponins with structurally different glycosylation or acylation patterns that affect their biological activities. Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation.

Saponins of Quillaja saponaria Molina in particular can have potent adjuvant activity, but also can be chemically unstable, show hemolytic activity, and be associated with immediate pain at injection sites. Saponin preparations based on defined compositions of purified saponin fractions of Quillaja saponaria Molina are described, for example, by Cox et al., PCT/AU1995/000670 (WO96011711).

Incorporation of saponins of Quillaja saponaria Molina into particles comprising saponin and lipid can attenuate the chemical instability, hemolytic activity, and immediate pain when injected associated with saponins. Specific examples of particles comprising saponin and lipid include saponin based particles and saponin based antigen-presenting particles, as taught, for example, by Stertman et al., Human Vaccines & Immunotherapeutics, 2023, 19(1):2189885, GSK's Liposome-based Adjuvant System 01 particles, as described, for example, in Didierlaurent et al., Expert Review of Vaccines, 2017, 16(1):55-63, and Army Liposome Formulation Q particles, as described, for example, by Alving et al., Expert Review of Vaccines, 2020, 19(3):279-292.

Novavax's Matrix particle adjuvant is a saponin-based adjuvant derived from Quillaja saponins, which are extracted from the bark of the Quillaja saponaria Molina tree. The bark extract is fractionated, and fractionated saponins are formulated with phosphatidylcholine and cholesterol into cage-like Matrix particles. Matrix adjuvant may be a formulation that is manufactured using two extracts of saponins of Quillaja saponaria Molina, termed saponin fraction A and saponin fraction C, which are described in detail below. To make a Matrix adjuvant, saponin fraction A and saponin fraction C are separately mixed with cholesterol and phosphatidylcholine, in the presence of the detergent Mega-10 to form dispersions of approximately 40-50 nm-sized stable cage-like structures, designated Matrix-A and Matrix-C particles. The Matrix-A particles may comprise fraction A but not fraction C, whereas the Matrix-C particles may comprise fraction C but not fraction A. The Mega-10 detergent is removed by diafiltration. In one embodiment, the Matrix-A to Matrix-C weight ratio is between 80:20 and 90:10. In some embodiments the Matrix-A to Matrix-C ratio is around 85:15. A specific embodiment, denoted Matrix-M, constitutes Matrix-A and Matrix-C particles at a fixed weight ratio of 85:15 of Matrix-A particles to Matrix-C particles.

Considering saponins in more detail, as noted above, saponin preparations based on defined compositions of purified saponin fractions of Quillaja saponaria Molina are described, for example, by Cox et al., PCT/AU1995/000670 (WO96011711).

Over the years the process for fractionation of saponin raw material into fraction A and fraction C has been developed and scaled-up. For example, as described in Cox et al., PCT/AU1995/000670 (WO96011711), fractions A, B, and C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. Fraction A is the fraction that is eluted at approximately 39% acetonitrile. Fraction C is the fraction that is eluted at approximately 49% acetonitrile.

As noted above, saponin preparations based on defined compositions of purified saponin fractions of Quillaja saponaria Molina are described, for example, by Cox et al., PCT/AU1995/000670 (WO96011711).

In specific embodiments, Saponin Fraction C has a purity of at least 80%, as determined by HPLC. In specific embodiments, Cholesterol is derived from a plant-based source (phytosterol). In specific embodiments, Phosphatidylcholine comprises POPC fatty acid chains. Matrix particles can be made as described, for example, in Morein et al. PCT/SE1989/000528, and Morein et al., PCT/SE/2003/001180. In specific embodiments, the Matrix A and Matrix C particles are in the form of a cage-like structure, which is confirmed by transmission electron microscopy.

### Formulations

The vaccine is prepared in a formulation for delivery to a subject, typically a human subject. The formulation may be aqueous-based, but it not so restricted. In some embodiments the vaccine, comprising the antigen and adjuvant, is in a buffered solution. For example, the buffer may include any pharmaceutically acceptable buffer, such as Tris or MES. The formulation may also include other excipients, such as sodium chloride (NaCl) and sucrose, and may also contain other excipients, e.g. arginine. A typical dose is delivered in an amount of 0.1-1 mL and may be 0.2 mL. The non-active excipients, i.e. those ingredients in the vaccine formulation not including the mRNA or adjuvant, are generally known.

A dose of mRNA vaccine for human administration includes a therapeutically effective amount of mRNA, likely in the range 0.1-100 μg, preferably in the range 1-50 μg, more preferably in the range 2-20 μg. In embodiments, the dose of mRNA is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 40 μg, about 50, about 60, about 70, about 80, about 90, or about 100 μg, or even higher, including all values and ranges in between. In embodiments, the dose of mRNA is 5 μg. In embodiments, the dose of mRNA glycoprotein protein is 10 μg. In some embodiments, the dose of mRNA is the same for an initial dose and for one or more subsequent boost doses, if required.

In embodiments, the vaccine includes a therapeutically effective amount of a saponin-based adjuvant in the range 0.5-200 μg, preferably in the range 10-100 μg. In embodiments, the dose of the adjuvant administered with mRNA is from about 1 μg to about 100 μg, for example, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of adjuvant. In embodiments, the dose of adjuvant is about 50 μg.

### Administration

In embodiments, the compositions may be used for the prevention and/or treatment of one or more of a SARS-CoV-2 infection, a heterogeneous SARS-CoV-2 strain infection, a SARS infection, a MERS infection, and influenza infection, or a combination thereof. Thus, the disclosure provides a method for eliciting an immune response against one or more of the SARS-CoV-2 virus, heterogeneous SARS-CoV-2 virus, MERS, SARS, and an influenza virus. The method involves administering an immunologically effective amount of a composition described herein to a subject. In some embodiments, the mRNA vaccine may include glycoprotein antigens against seasonal influenza. Advantageously, the compositions disclosed herein induce particularly useful anti-coronavirus and/or anti-influenza responses.

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

The compositions disclosed herein may be stored in a pre-filled syringe. In other embodiments, the compositions may be provided in a kit that has a first vial containing the antigen and a second vial containing the adjuvant, for example a Matrix-M adjuvant. In such a case, one of the vials has sufficient empty space to receive the contents of the other vial. The first vial of the kit may contain the antigen in a lyophilized form.

In some embodiments, the mRNA vaccine is administered to an individual on just one occasion. In other embodiments, the vaccine is administered to an individual on at least two different occasions. The individual may be administered a first dose of the vaccine and then administered a second dose a certain amount of time later. In embodiments, a second dose may be administered up to one week, two weeks, three weeks, four weeks, six weeks, two months, three months, four months, five months, six months, seven months, eight months, nine months, ten months, eleven months or twelve months after the initial dose. In other embodiments a second dose may be administered within a range of time after the initial dose, for example within one week to one month, two months, three months, four months, five months, six months, seven months, eight months, nine months, ten months, eleven months or twelve months after the initial dose. In other embodiments a second dose may be administered within one month to two months, three months, four months, five months, six months, seven months, eight months, nine months, ten months, eleven months or twelve months after the initial dose. In other embodiments a second dose may be administered within two months to three months, four months, five months, six months, seven months, eight months, nine months, ten months, eleven months or twelve months after the initial dose. In other embodiments a second dose may be administered within three months and four months, five months, six months, seven months, eight months, nine months, ten months, eleven months or twelve months after the initial dose. In other embodiments a second dose may be administered within six months and seven months, eight months, nine months, ten months, eleven months or twelve months after the initial dose.

This invention is further illustrated by the following examples that should not be construed as limiting. The contents of all references, patents and published patent applications cited throughout this application, as well as the Figures and the Sequence Listing, are incorporated herein by reference for all purposes.

### Examples

### Example 1: Study of the Effect of Matrix-M on Commercial mRNA SARS-CoV-2 Vaccine

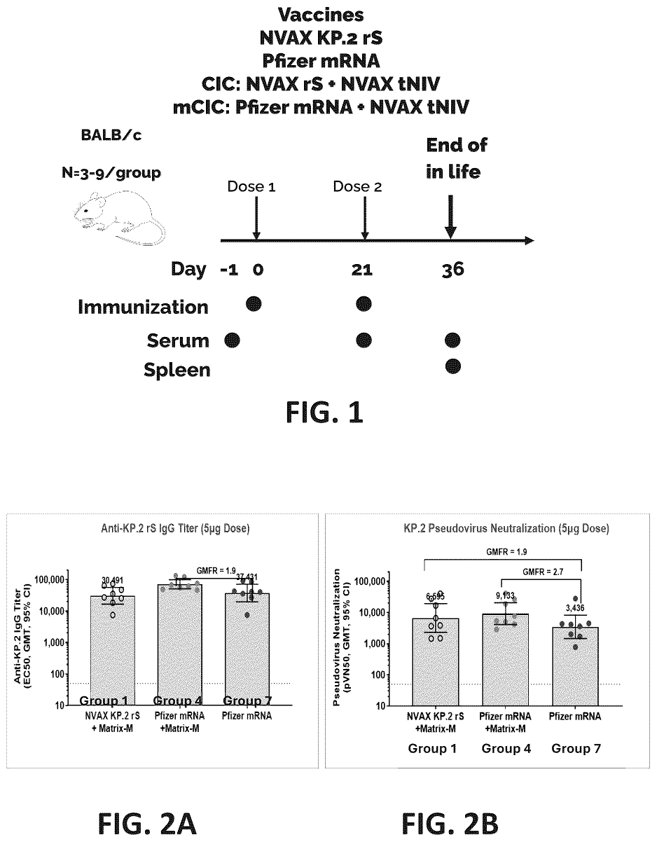

A study was made comparing the immunogenic effect in mice of administering a commercially available SARS-CoV-2 mRNA vaccine with and without Matrix-M adjuvant. The timeline of the study is shown in FIG. 1. BALB/c were administered the vaccine on Days 0 and 21 (Week 3) of the study. Serum was taken from the mice on Day −1 (the day before Day 0) and just prior to the administration of dose 2 of the vaccine. Serum was also taken on Day 36, at which point the mice were sacrificed to obtain their spleens. Serum was used in IgG titer assays and in pseudovirus neutralization pVNT₅₀ titer assays. Spleen tissue was used in the measurement of CD4+ and CD8+ T cell responses.

The table below shows what each mouse group was administered. There were 8 mice in each of Groups 1-9, 9 mice in each of Groups 10 and 11, and 3 mice in the placebo group, Group 12.

TABLE 1
Combinations of vaccine and adjuvant used in Example 1

| | | | Matrix-M |
|---|---|---|---|
| Group | Vaccines | Vaccine dose | dose |
| No. | (Days 0 and 21) | (μg) | (μg) |
| 1 | NVAX: KP.2 rS Protein | 5 | 5 |
| 2 | NVAX: KP.2 rS Protein | 1 | 5 |
| 3 | NVAX: KP.2 rS Protein | 0.2 | 5 |
| 4 | Pfizer: KP.2 mRNA | 5 | 5 |
| 5 | Pfizer: KP.2 mRNA | 1 | 5 |
| 6 | Pfizer: KP.2 mRNA | 0.2 | 5 |
| 7 | Pfizer: KP.2 mRNA | 5 | — |
| 8 | Pfizer: KP.2 mRNA | 1 | — |
| 9 | Pfizer: KP.2 mRNA | 0.2 | — |
| 10 | NVAX: KP.2 rS Protein + | 1 (NVAX rS) | 5 |
| | NVAX: tNIV | 4.5 (NVAX tNIV) | |
| 11 | Pfizer: KP.2 mRNA + | 1 (Pfizer mRNA) | 5 |
| | NVAX: tNIV | 4.5 (NVAX tNIV) | |
| 12 | Placebo | n/a | |

NVAX: KP.2 rS refers to a recombinant spike protein vaccine for the SARS-CoV-2 KP.2 variant, available from Novavax Inc., Gaithersburg, MD. Pfizer: KP.2 refers to an mRNA vaccine for the SARS-CoV-2 KP.2 variant, available from Pfizer Inc., New York, NY. As is shown in the table, when administered alone, the NVAX: KP.2 rS vaccine and the Pfizer: KP.2 mRNA vaccines were administered at 0.2, 1 or 5 μg. When administered alone, the Pfizer: KP.2 vaccine was either administered with or without Matrix-M adjuvant. Two groups, 10 and 11, included a SARS-CoV-2 KP.2 vaccine combined with a Novavax recombinant protein tetravalent seasonal influenza vaccine, NVAX: tNIV. In each case, the SARS-CoV-2 KP.2 vaccine was present in an amount of 1 μg, and the tetravalent influenza vaccine was present in an amount of 4.5 μg. When included in the vaccine, Matrix-M was present in an amount of 5 μg.

KP.2 rS was diluted to target concentration in 25 mM NaPi, 300 mM NaCl, pH 7.2, 0.01% PS80 buffer and mixed with Matrix-M for standalone groups. KP.2 mRNA was prepared by diluting to target concentration in phosphate-buffered saline (PBS) buffer with and without Matrix-M for standalone groups. For combination groups, tNIV was added to standalone KP.2 rS or Pfizer KP.2 mRNA. PBS was used for placebo.

The spike protein enzyme-linked immunosorbent assay (ELISA) was used to determine anti-SARS-CoV-2 spike (S) protein immunoglobulin G (anti-S IgG) titers. Briefly, 96-well microtiter plates (Thermo Fisher Scientific, Rochester, NY, USA) were coated with 1.0 μg/mL of SARS-CoV-2 rS protein. Plates were washed with PBS-T and non-specific binding was blocked with TBS Startblock blocking buffer (Thermo Fisher Scientific). Mouse serum samples were serially diluted 3-fold starting with a 1:100 dilution (i.e., 10⁻² to 10⁻⁸) and added to the coated plates, followed by incubation at room temperature for 2 hours. Following incubation, plates were washed with PBS-T and HRP-conjugated goat anti-mouse IgG (Southern Biotech, Birmingham, AL, USA) was added for 1 hour. Plates were washed with PBST and 3,3′,5,5′-tetramethylbenzidine (TMB) peroxidase substrate (Sigma, St. Louis, MO, USA) was added.

Reactions were stopped with TMB stop solution (ScyTek Laboratories, Inc. Logan, UT). Plates were read at an optical density (OD) of 450 nm with a SpectraMax Plus plate reader (Molecular Devices, Sunnyvale, CA, USA). Effective concentration at 50% (EC₅₀) titers were calculated by 4-parameter fitting using SoftMax Pro 6.5.1 GxP software. Individual animal anti-S IgG titers, group geometric mean titers (GMTs), and 95% confidence intervals (±95% CI) were plotted using GraphPad Prism 10 software. For a titer below the assay lower limit of detection (LOD), a titer of <100 (starting dilution) was reported and a value of “50” assigned to the sample to calculate the group GMT.

The pseudovirus neutralization assay was performed using a HEK293T cell line stably expressing hACE2 (HEK293T/ACE2 obtained from Creative Biogene). Heat-inactivated mice serum samples were serially diluted three-fold beginning at 1:50 (Day 35) in reduced serum Opti-MEM. Pseudovirus was diluted in reduced serum Opti-MEM at the appropriate virus dilution previously determined in a virus titration. Briefly, pseudovirus was serially diluted 2-fold following assay methods and a dilution targeting 100,000 RLU (range 50,000-250,000) within the linear range was targeted. Fifty microliters of diluted sera in a 96-well plate was incubated with 50 μL of diluted pseudovirus for 1 hour at 37° C., 5% CO₂. Control wells including cell only and pseudovirus only wells are included on each plate. Following incubation, HEK293T cells expressing ACE2 were resuspended in cell culture media (DMEM without phenol red+5% FBS +1% Penicillin+streptomycin+glutamine, with 1.25 μg/mL puromycin) and 100 μL of 2.0×10⁴ cells/mL was added to each well in the 96well tissue culture plate. Plates were incubated for 72 hours at 37° C. Plates were removed from the incubator to acclimate to <25° C. prior to addition of BrightGlo Luciferase Substrate (Promega). Fifty microliters of BrightGlo Luciferase was added to each well without ambient light. Plates were incubated for five minutes in the dark prior to reading on an iD3 spectrophotometer (SpectraMax) measuring luminescence. Plates were read within 25 minutes of addition of substrate. At least one positive and negative control monoclonal antibody was included for each pseudovirus tested each day. Data were analyzed and neutralization curves were generated in GraphPad Prism for each sample, 50% pseudovirus Neutralization Titers (pVN₅₀) were calculated by 4-parameter curve fitting.

Antigen-specific T cell response was measured by Intracellular Cytokine staining assay. Cells were cultured in a 96-well U-bottom plate at 2×10⁶ cells per well. The cells were stimulated with JN.1, JN.1.13.1, or KP.2 or XEC Omicron rS, or JN.1 S Peptide Pool (pooled peptides of 15 amino acids in length and overlapping by 11 amino acids that cover the entire S protein; divided into two pools for testing purposes). The plate was incubated 6 h at 37° C. in the presence of BD GolgiPlug™ and BD GolgiStop™ (BD Biosciences) for the last 4 h of incubation. Cells were labeled with murine antibodies against CD3 (BV650), CD4 (APC-H7), CD8 (FITC), CD44 (Alexa Fluor 700), and CD62L (PE) (BD Pharmingen, CA) and the yellow LIVE/DEAD® dye. After fixation with Cytofix/Cytoperm (BD Biosciences), cells were incubated with PerCP-Cy5.5-conjugated anti-IFN-γ, BV421-conjugated anti-IL-2, PE-cy7-conjugated anti-TNF-α, and APC-conjugated anti-IL-4 (BD Biosciences). All stained samples were acquired using an LSR-Fortessa or a FACSymphony flow cytometer (Becton Dickinson, San Jose, CA) and the data were analyzed with FlowJo software version Xv10 (Tree Star Inc., Ashland, OR). All analyzed data were gated on effector CD4+ T cells (CD44^{hi} CD62L^{low}CD4⁺) or effector CD8⁺ T cells (CD44^{hi} CD62L^{low}CD8⁺), and the data are shown as the number of cytokine secreting cells per million effector CD4⁺ T cells or CD8⁺ T cells. The lower limit of detection (LOD) for the ICCS assay of CD4⁺ T cells is 10 for all cytokines except for TNF-α (LOD of 100). The LOD for the ICCS assay of CD8⁺ T cells is 100.

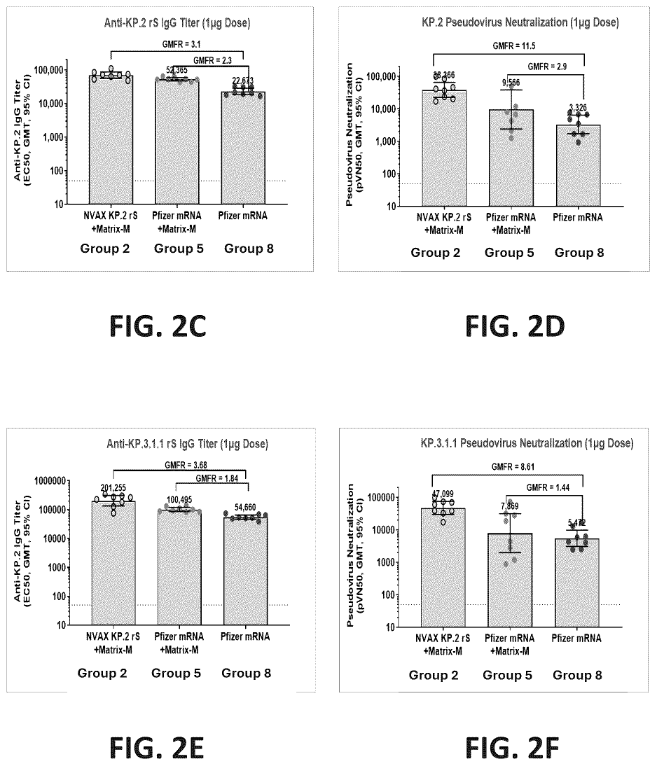

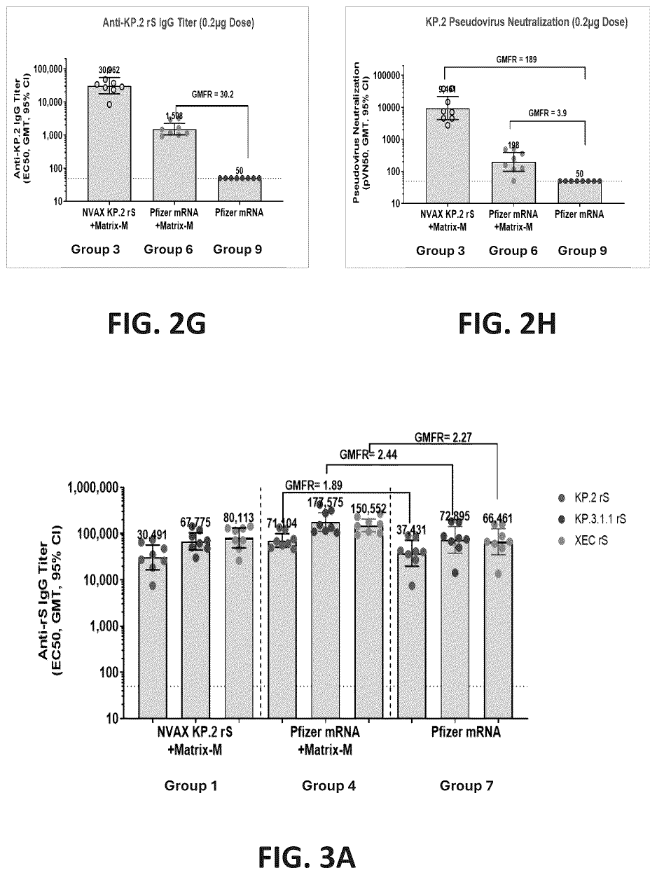

FIGS. 2A and 2B respectively present the IgG titer and pseudovirus neutralization titer results for the 5 μg single vaccine doses (Groups 1, 4, and 7). In FIG. 2A, the y-axis is anti-KP.2 IgG titer (EC₅₀, GMT, 95% Confidence interval). The IgG titer for the protein vaccine (Group 1) was about the same as for the mRNA vaccine without Matrix-M (Group 7), ~30,500 v. 37,500. However, the response to the mRNA vaccine with Matrix-M (Group 4) was unexpectedly high ~around 1.9 times that of the mRNA vaccine without Matrix-M (Group 7). In FIG. 2B, the y-axis is pseudovirus neutralization (pVN₅₀, GMT, 95% confidence interval). The results of the neutralization titer for the protein vaccine (Group 1) were about 1.9 times higher than the mRNA vaccine without Matrix-M (Group 7), ~6,600 v. 3,400. Again, the response to the mRNA vaccine with Matrix-M (Group 4) was unexpectedly high—around 2.7 times that of the mRNA vaccine without Matrix-M (Group 7), ~9,100 v. 3,400).

FIGS. 2C and 2D respectively show the same results as FIGS. 2A and 2B, except for the 1 g doses (Groups 2, 5, and 8). The IgG titer for the protein vaccine (Group 2), as shown in FIG. 2C, was about three times higher than the mRNA vaccine without Matrix-M (Group 8). The response to the mRNA vaccine with Matrix-M (Group 5) was higher than that of the mRNA vaccine without Matrix-M (Group 8), about 52,000 v. about 23,000. The results of the neutralization titer, as shown in FIG. 2D, for the protein vaccine (Group 2) were about 11.5 times higher than the mRNA vaccine without Matrix-M (Group 8), ~38,400 v. ~3,300. The response to the mRNA vaccine with Matrix-M (Group 5) was around 2.9 times that of the mRNA vaccine without Matrix-M (Group 8), ~9,600 v. 3,300).

Cross-immunization capability at the 1 μg dose is illustrated in FIGS. 2E and 2F. FIG. 2E shows the results of an anti-KP.3.1.1.rS IgG titer. The IgG titer for the protein vaccine (Group 2), as shown in FIG. 2E, was about 3.7 times higher than the mRNA vaccine without Matrix-M (Group 8), ~201,000 v. ~55,000. The IgG response to the mRNA vaccine with Matrix-M (Group 5) was about 1.8 times higher than that of the mRNA vaccine without Matrix-M (Group 8), ~100,000 v. 55,000. The results of the pseudovirus neutralization titer, as shown in FIG. 2F, for the protein vaccine (Group 2) were about 8.6 times higher than the mRNA vaccine without Matrix-M (Group 8), ~47,000 v. ~5,400. The response to the mRNA vaccine with Matrix-M (Group 5) was around 1.4 times that of the mRNA vaccine without Matrix-M (Group 8), ~7,900 v. 5,400).

FIGS. 2G and 2H respectively show the same results as FIGS. 2A and 2B, except for the 0.2 μg doses (Groups 3, 6, and 9). The IgG titer for the protein vaccine (Group 3), as shown in FIG. 2G, was at least 620 times higher than the mRNA vaccine without Matrix-M (Group 9), ~31,000 v. 50, the LOD. The response to the mRNA vaccine with Matrix-M (Group 6) was about 30 times higher than that of the mRNA vaccine without Matrix-M (Group 9), ~1,500 v. ~50. The results of the neutralization titer, as shown in FIG. 2H, for the protein vaccine (Group 3) were about 190 times higher than the mRNA vaccine without Matrix-M (Group 9), ~9,500 v. ~50. The response to the mRNA vaccine with Matrix-M (Group 6) was around four times that of the mRNA vaccine without Matrix-M (Group 9), ~200 v. 50).

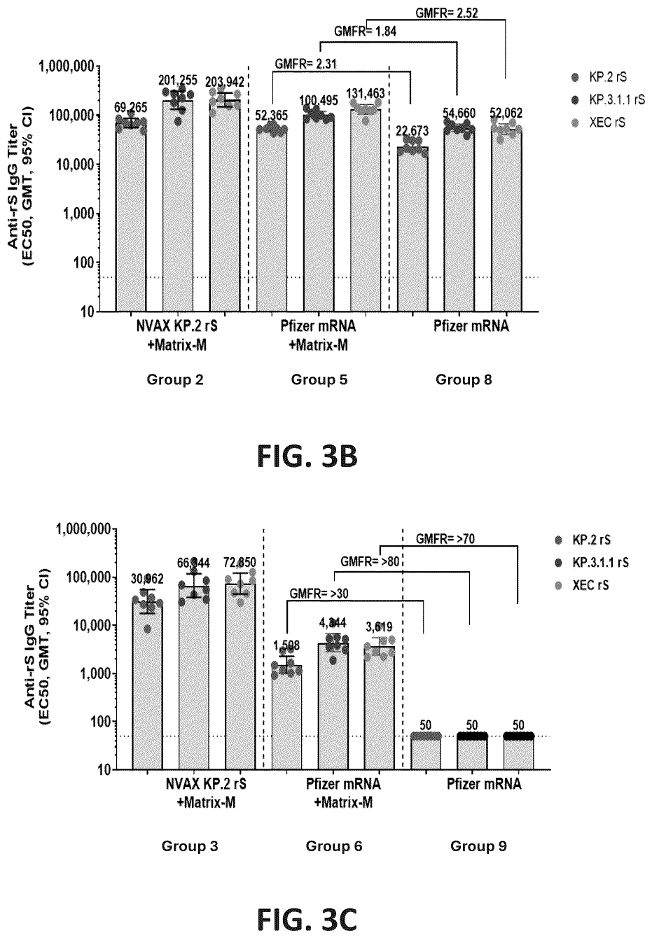

FIGS. 3A-3C further illustrate the breadth of the immunization spectrum arising from each of the single vaccine doses. Three bars are shown for each Group corresponding, in order from left to right, to i) anti-KP.2 rS IgG, ii) anti-KP.3.1.1 IgG, and iii) anti-XEC rS IgG. FIG. 3A shows the results of anti-rS IgG titers for the 5 μg dose (Groups 1, 4, and 7). The IgG titers for Group 4 (mRNA+Matrix-M) are all higher than those in Group 7 (mRNA alone), with the KP.2 rS IgG titer being higher by about 1.9 fold, the KP.3.1.1 rS IgG titer being higher by about 2.4 fold, and the XEC rS IgG titer by about 2.3 fold.

FIG. 3B shows the results of anti-rS IgG titers for the 1 μg dose (Groups 2, 5, and 8). The IgG titers for Group 5 (mRNA+Matrix-M) are all higher than those in Group 8 (mRNA alone), with the KP.2 rS IgG titer being higher by about 2.3 fold, the KP.3.1.1 rS IgG titer being higher by about 1.8 fold, and the XEC rS IgG titer by about 2.5 fold. Additionally, the IgG titers for Group 2 (NVAX KP.2 rS+Matrix-M) were all higher than the respective Group 8 titers.

FIG. 3C shows the results of anti-rS IgG titers for the 0.2 μg dose (Groups 3, 6, and 9). The IgG titers for Group 6 (mRNA+Matrix-M) are all higher than those in Group 9 (mRNA alone), which were below the lowest level of detection (LOD), the KP.2 rS IgG titer being higher by at least 30 fold, the KP.3.1.1 rS IgG titer higher by at least 80 fold, and the XEC rS IgG titer by at least 70 fold. Additionally, the IgG titers for Group 3 (NVAX KP.2 rS+Matrix-M) were all higher than the respective Group 9 titers.

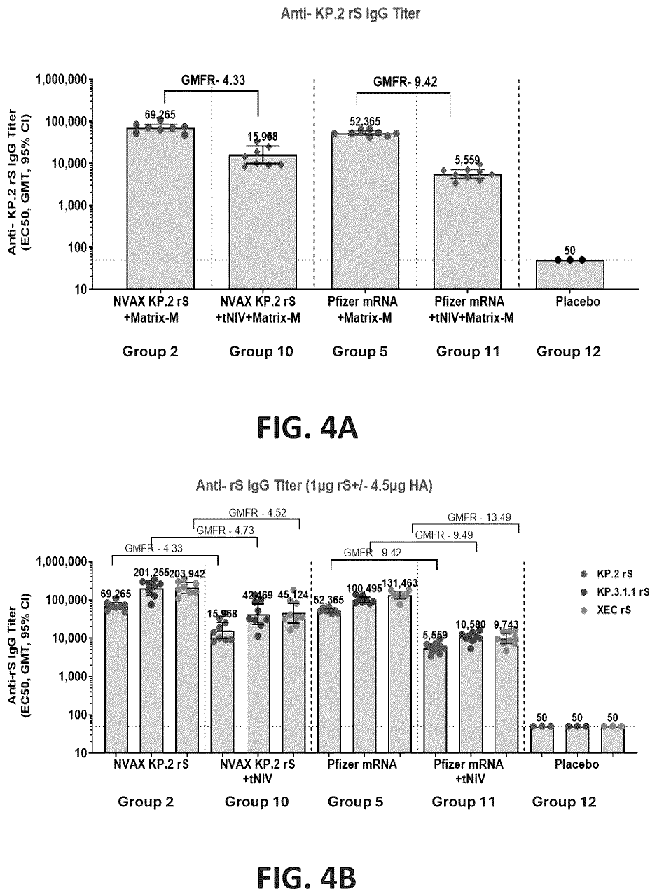

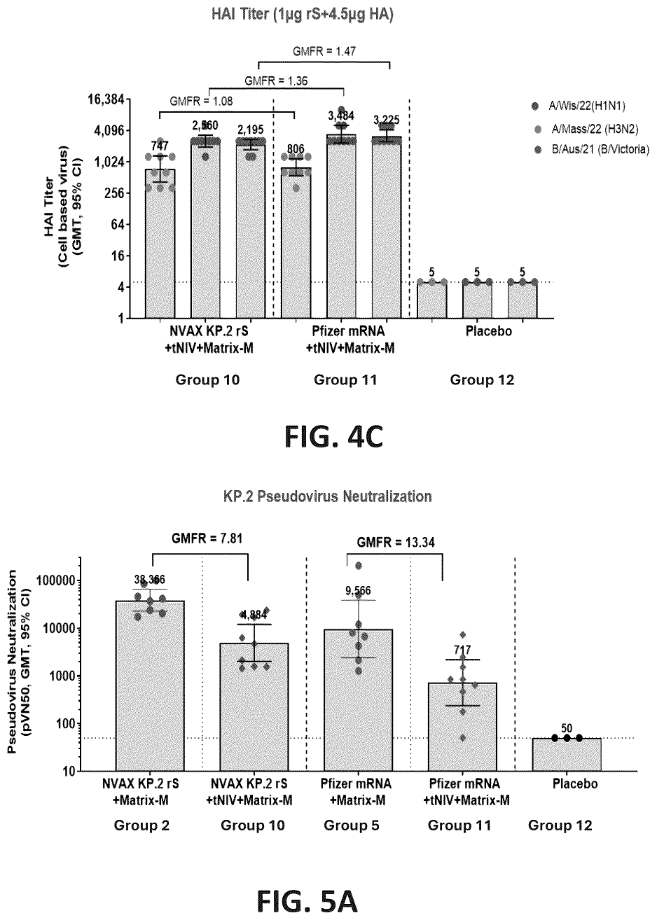

FIGS. 4A-4C present results comparing the performance of the 1 μg doses (Groups 2 and 5) with the doses that included the tetravalent seasonal influenza vaccine (Groups 10 and 11), along with the placebo. FIG. 4A presents anti-KP.2 rS IgG titer results for Groups 2, 5, 10, 11, and placebo. The y-axis shows anti-KP.2 IgG titer (EC₅₀, GMT, 95% Confidence interval). For both the rS protein vaccine and the mRNA vaccine, the titers were higher without the tetravalent influenza vaccine than with. In particular, the response in Group 2 mice (NVAX KP.2 rS+Matrix-M) was around 4.3 times that in Group 10 mice (NVAX KP.2 rS+tNIV+Matrix-M). Also, the response in Group 5 mice (mRNA+Matrix-M) was around 9.4 times that in Group 11 mice ((mRNA+tNIV+Matrix-M).

FIG. 4B shows results of different anti-rS IgG titers, illustrating the breadth of the immunization spectrum arising from each of the vaccine doses. Three bars are shown for each Group corresponding, in order from left to right, to i) anti-KP.2 rS IgG, ii) anti-KP.3.1.1 IgG, and iii) anti-XEC rS IgG. For both the rS protein vaccine and the mRNA vaccine, the different IgG titers produced higher results when the tetravalent influenza vaccine was absent.

FIG. 4C shows the results of hemagglutinin influenza (HAI) titers for Groups 10 (NVAX KP.2+tNIV+Matrix-M), 11 (mRNA+tNIV+Matrix-M), and 12 (placebo). Three bars are shown for each Group corresponding, in order from left to right, to i) A/Wis/22 (H1N1), ii) A/Mass/22 (H3N2), and iii) B/Aus/21 (B/Victoria) influenza strains. The titers for the protein vaccine (Group 10) were around the same as, or slightly higher than, the mRNA vaccine (Group 11).

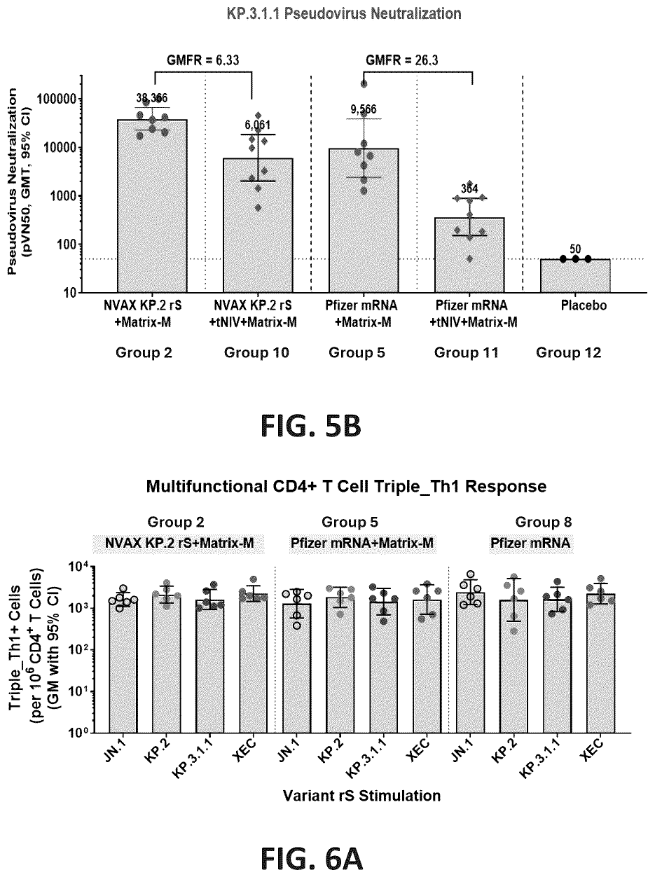

FIGS. 5A and 5B show the results of pseudovirus neutralization assays for Groups 2, 5, 10, 11, and 12. FIG. 5A shows KP.2 pseudovirus neutralization results. For the NVAZ KP.2 rS vaccine, the assay showed higher neutralization when the tNIV was absent compared to when the tNIV was present, by a factor of about 7.8. Likewise, for the mRNA vaccine, the assay showed higher neutralization when the tNIV was absent compared to when the tNIV was present, by a factor of about 13.3. FIG. 5B illustrates the breadth of the immunological effect with a KP.3.1.1 pseudovirus neutralization. For the NVAZ KP.2 rS vaccine, the assay showed higher neutralization when the tNIV was absent compared to when the tNIV was present, by a factor of about 6.3. For the mRNA vaccine, the assay showed higher neutralization when the tNIV was absent compared to when the tNIV was present, by a factor of about 13.3.

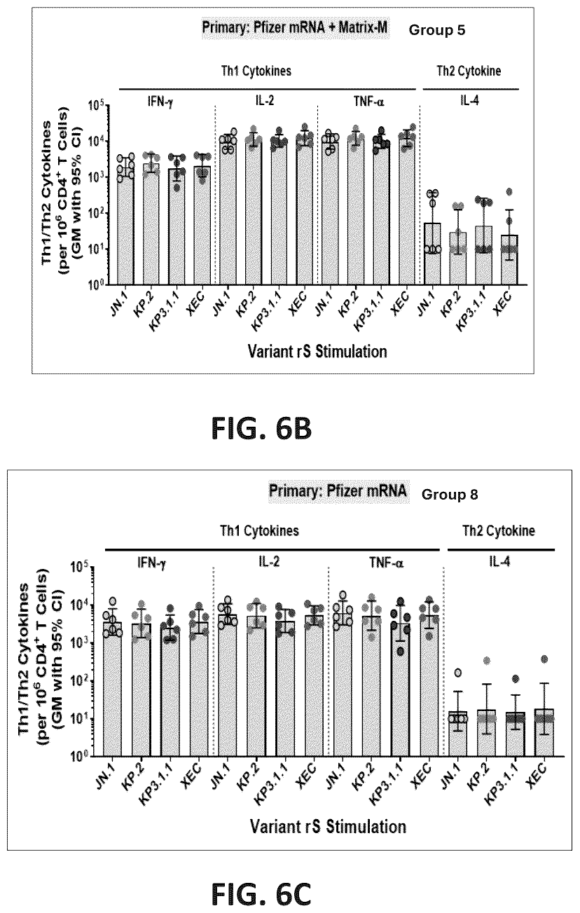

FIGS. 6A-6C illustrate T cell responses from spleen tissue for Groups 2, 5, and 8, using an intracellular cytokine staining (ICCS assay). FIG. 6A shows the triple Th1 CD4+ T cell response for Groups 2, 5, and 8. The units on the y-axis are the number of triple Th1+ cells per 10⁶ CD4+ T cells. The response for each Group is shown as four bars representing, in order, stimulation with JN.1, KP.2, KP.3.1.1, and XEC strains of SARS-CoV-2. FIG. 6B shows cytokine count for four different cytokines, IFN-γ, IL2, TNF-α, and IL4, with stimulation by the same four SARS-CoV-2 strains, for Group 5 (mRNA+Matrix-M). FIG. 6C shows cytokine count for the four different cytokines, IFN-γ, IL2, TNF-α, and IL4, with stimulation by the same four SARS-CoV-2 strains, for Group 8 (mRNA). A comparison between FIGS. 6B and 6C shows that the response of IFN-7 is higher for Group 8, while the responses for IL2, TNF-α, and IL4 are generally higher for Group 5.

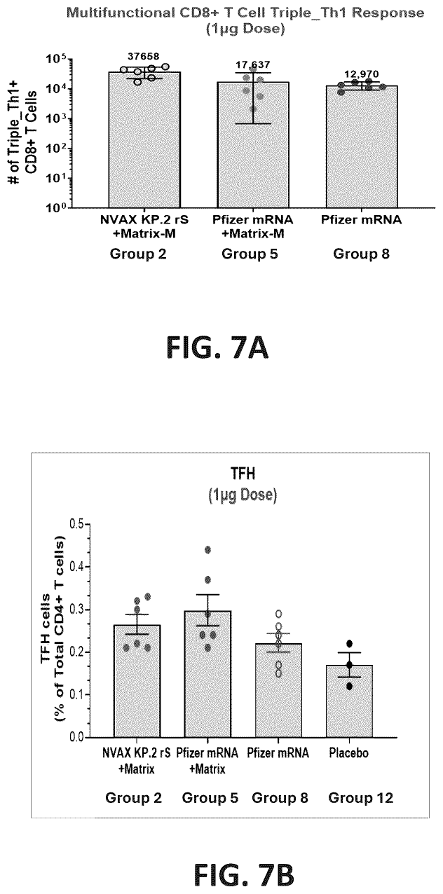

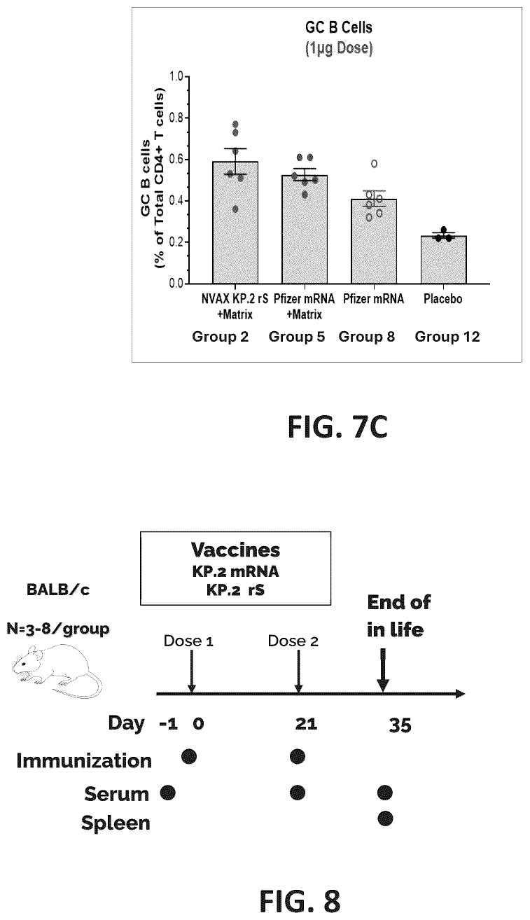

FIG. 7A shows the multifunctional CD8+ T cell triple Th1 response for groups 2, 5, and 8. The units on the y-axis are number of triple Th1+CD8+ cells. The response was higher for Group 2 than Group 5, 37,658 v. 17,637. The response for Group 5 was higher than the response for Group 8, 17,637 v. 12,970, representing an increase of 1.36 times when the mRNA vaccine was administered with Matrix-M.

FIG. 7B shows the follicular B helper T (TFH) cell response for Groups 2, 5, 9, and 12. The units on the y-axis are TFH cells as a percentage of the total CD4+ T cells. The highest response was for Group 5 (mRNA+Matrix-M). FIG. 7C shows the numbers of germinal center B cells (GC B cells) for Groups 2, 5, 9, and 12. The units on the y-axis are % of total CD4+ T cells. The highest response is by Group 2. Group 5 shows a higher response than Group 8.

### Example 2: Study of the Effect of Matrix-M on Commercial mRNA SARS-CoV-2 Vaccine

Another study was made comparing the immunogenic effect in mice of administering two different commercially available SARS-CoV-2 mRNA vaccines with and without Matrix-M adjuvant. The timeline of the study is shown in FIG. 8. BALB/c were administered the vaccine on Days 0 and 21 (Week 3) of the study. Serum was taken from the mice on Day −1 (the day before Day 0) and just prior to the administration of dose 2 of the vaccine. Serum was also taken on Day 36, at which point the mice were sacrificed to obtain their spleens. Serum was used in IgG titer assays and in pseudovirus neutralization pVNT₅₀ titer assays. Spleen tissue was used in the measurement of CD4+ and CD8+ T cell responses.

The table below shows what each mouse group was administered. There were 8 mice in each of Groups 1-12 and 3 mice in the placebo group, Group 13.

TABLE 2
Combinations of vaccine and adjuvant used in Example 2

| | | | Matrix-M |
|---|---|---|---|
| Group | Vaccines | Vaccine dose | dose |
| No. | (Days 0 and 21) | (μg) | (μg) |
| 1 | Pfizer: KP.2 mRNA | 1 | — |
| | (COMIRNATY) | | |
| 2 | Pfizer: KP.2 mRNA | 0.5 | — |
| | (COMIRNATY) | | |
| 3 | Pfizer: KP.2 mRNA | 0.2 | — |
| | (COMIRNATY) | | |
| 4 | Moderna: KP.2 mRNA | 1 | — |
| | (SPIKEVAX) | | |
| 5 | Moderna: KP.2 mRNA | 0.2 | — |
| | (SPIKEVAX) | | |
| 6 | Pfizer: KP.2 mRNA | 1 | 5 |
| | (COMIRNATY) | | |
| 7 | Pfizer: KP.2 mRNA | 0.5 | 5 |
| | (COMIRNATY) | | |
| 8 | Pfizer: KP.2 mRNA | 0.2 | 5 |
| | (COMIRNATY) | | |
| 9 | Moderna: KP.2 mRNA | 1 | 5 |
| | (SPIKEVAX) | | |
| 10 | Moderna: KP.2 mRNA | 0.2 | 5 |
| | (SPIKEVAX) | | |
| 11 | NVAX: KP.2 rS Protein | 1 | 5 |
| 12 | NVAX: KP.2 rS Protein | 0.2 | 5 |
| 13 | Placebo | | |

Pfizer: KP.2 is an mRNA vaccine for the SARS-CoV-2 KP.2 variant, available from Pfizer Inc., New York, NY, under the brand name COMIRNATY. The Pfizer KP.2 vaccine was administered without Matrix-M in 1 ag, 0.5 ag, and 0.2 ag doses (Groups 1, 2, and 3, respectively), and with 5 ag Matrix-M in 1 ag, 0.5 ag, and 0.2 ag doses (Groups 6, 7, and 8, respectively). Moderna KP.2 is an mRNA vaccine for the SARS-CoV-2 KP.2 variant, available from Moderna Inc., Cambridge, MA, under the brand name SPIKEVAX. Moderna KP.2 was administered without Matrix-M in 1 ag and 0.2 ag doses (Groups 4 and 5, respectively) and with 5 ag Matrix-M in 1 ag and 0.2 ag doses (Groups 9 and 10, respectively). NVAX: KP.2 rS refers to the same Novavax recombinant spike protein vaccine for the SARS-CoV-2 KP.2 variant used in Example 1. NVAX: KP.2 was administered with 5 μg Matrix-M in 1 μg and 0.2 μg doses (Groups 11 and 12, respectively).

MRNA Vaccination doses were prepared KP.2 mRNA Comirnaty and Spikevax were diluted in 1×PBS, with and without Matrix-M. KP2 rS was diluted in 25 mM NaPi, 300 mM NaCl, pH 7.2, 0.01% PS80 with Matrix-M.

The spike protein enzyme-linked immunosorbent assay (ELISA) was used to determine anti-SARS-CoV-2 spike (S) protein immunoglobulin G (anti-S IgG) titers. 96-well microtiter plates (Thermo Fisher Scientific, Rochester, NY, USA) were coated with 1.0 μg/mL of SARS-CoV-2 rS protein. Plates were washed with PBS-T and non-specific binding was blocked with TBS Startblock blocking buffer (Thermo Fisher Scientific). Mouse serum samples were serially diluted 3-fold starting with a 1:100 dilution (ie, 10⁻² to 10⁻⁸) and added to the coated plates, followed by incubation at room temperature for 2 hours. Following incubation, plates were washed with PBS-T and HRP-conjugated goat anti-mouse IgG (Southern Biotech, Birmingham, AL, USA) was added for 1 hour. Plates were washed with PBST and 3,3′,5,5′-tetramethylbenzidine (TMB) peroxidase substrate (Sigma, St. Louis, MO, USA) was added. Reactions were stopped with TMB stop solution (ScyTek Laboratories, Inc. Logan, UT). Plates were read at an optical density (OD) of 450 nm with a SpectraMax Plus plate reader (Molecular Devices, Sunnyvale, CA, USA). Effective concentration at 50% (EC₅₀) titers were calculated by 4-parameter fitting using SoftMax Pro 6.5.1 GxP software. Individual animal anti-S IgG titers, group geometric mean titers (GMTs), and 95% confidence intervals (±95% CI) were plotted using GraphPad Prism 10 software. For a titer below the assay lower limit of detection (LOD), a titer of <100 (starting dilution) was reported and a value of “50” assigned to the sample to calculate the group GMT.

The pseudovirus neutralization assays was performed using a HEK293T cell line stably expressing hACE2 (HEK293T/ACE2 obtained from Creative Biogene). Heat-inactivated mice serum samples were serially diluted three-fold beginning at 1:50 (Day 35) in reduced serum Opti-MEM. Pseudovirus was diluted in reduced serum Opti-MEM at the appropriate virus dilution previously determined in a virus titration. Briefly, pseudovirus was serially diluted 2-fold following assay methods and a dilution targeting 100,000 RLU (range 50,000-250,000) within the linear range was targeted. Fifty microliters of diluted sera in a 96-well plate was incubated with 50 μL of diluted pseudovirus for 1 hour at 37° C., 5% CO₂. Control wells including cell only and pseudovirus only wells are included on each plate. Following incubation, HEK293T cells expressing ACE2 were resuspended in cell culture media (DMEM without phenol red+5% FBS+1% Penicillin+streptomycin+glutamine, with 1.25 μg/mL puromycin) and 100 μL of 2.0×10⁴ cells/mL was added to each well in the 96well tissue culture plate. Plates were incubated for 72 hours at 37° C. Plates were removed from the incubator to acclimate to <25° C. prior to addition of BrightGlo Luciferase Substrate (Promega). Fifty microliters of BrightGlo Luciferase was added to each well without ambient light. Plates were incubated for five minutes in the dark prior to reading on an iD3 spectrophotometer (SpectraMax) measuring luminescence. Plates were read within 25 minutes of addition of substrate. At least one positive and negative control monoclonal antibody was included for each pseudovirus tested each day. Data were analyzed and neutralization curves were generated in GraphPad Prism for each sample, 50% pseudovirus Neutralization Titers (pVN₅₀) were calculated by 4-parameter curve fitting.

Antigen-specific T cell response was measured by Intracellular Cytokine staining assay. Briefly, cells were cultured in a 96-well U-bottom plate at 2×10⁶ cells per well. The cells were stimulated with JN.1, KP.2, XEC, LF.7, LP.8.1 Omicron rS, or JN.1 S Peptide Pool (pooled peptides of 15 amino acids in length and overlapping by 11 amino acids that cover the entire S protein; divided into two pools for testing purposes). The plate was incubated 6 h at 37° C. in the presence of BD GolgiPlug™ and BD GolgiStop™ (BD Biosciences) for the last 4 h of incubation. Cells were labeled with murine antibodies against CD3 (BV650), CD4 (APC-H7), CD8 (FITC), CD44 (Alexa Fluor 700), and CD62L (PE) (BD Pharmingen, CA) and the yellow LIVE/DEAD® dye. After fixation with Cytofix/Cytoperm (BD Biosciences), cells were incubated with PerCP-Cy5.5-conjugated anti-IFN-γ, BV421-conjugated anti-IL-2, PE-cy7-conjugated anti-TNF-α, and APC-conjugated anti-IL-4 (BD Biosciences). All stained samples were acquired using an LSR-Fortessa or a FACSymphony flow cytometer (Becton Dickinson, San Jose, CA) and the data were analyzed with FlowJo software version Xv10 (Tree Star Inc., Ashland, OR). All analyzed data were gated on effector CD4⁺ T cells (CD44^{hi} CD62L^{low}CD4⁺) or effector CD8⁺ T cells (CD44^{hi} CD62L^{low}CD8⁺), and the data are shown as the number of cytokine secreting cells per million effector CD4⁺ T cells or CD8⁺ T cells. The lower limit of detection (LOD) for the ICCS assay of CD4⁺ T cells is 10 for all cytokines except for TNF-α (LOD of 100). The LOD for the ICCS assay of CD8⁺ T cells is 100.

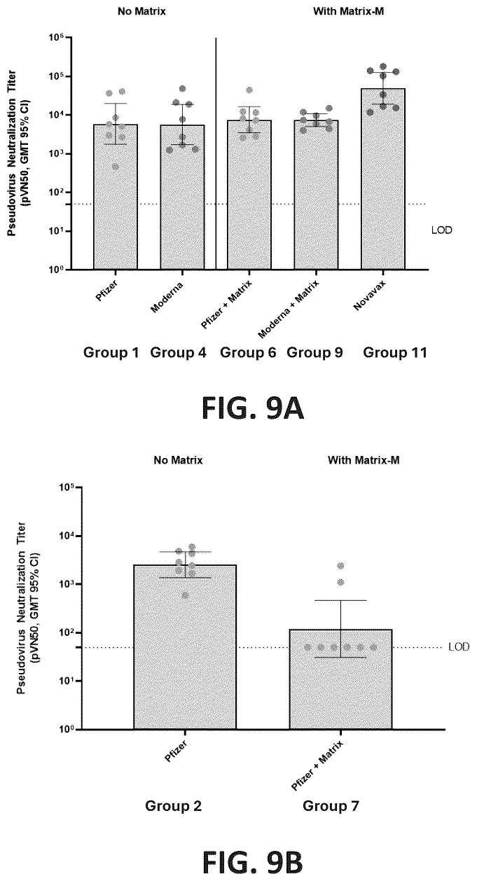

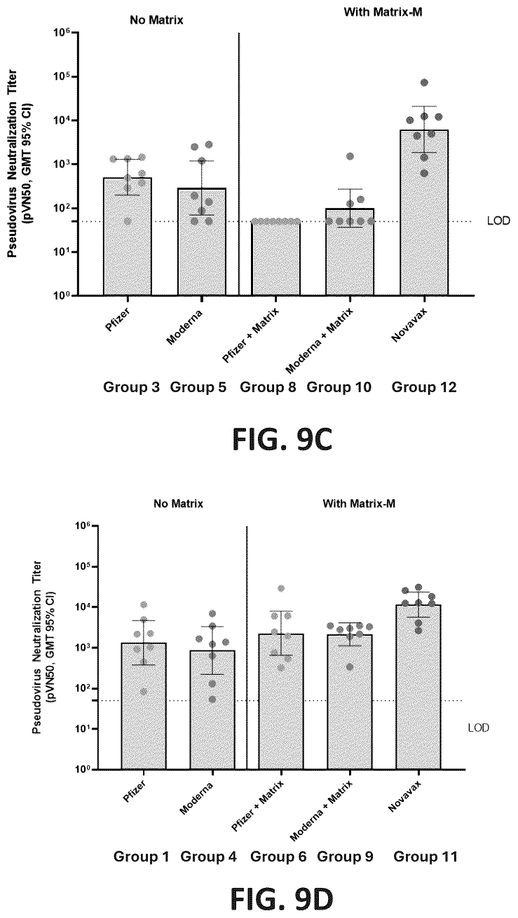

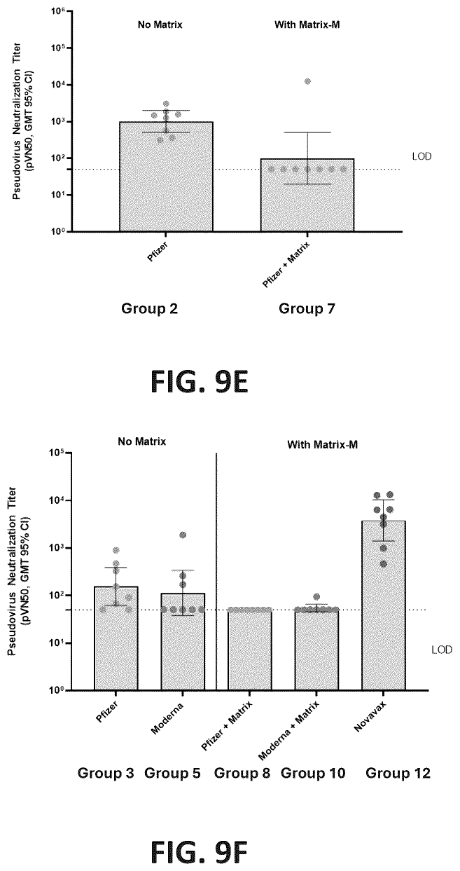

FIG. 9A shows homologous (KP.2) pseudovirus neutralization results for Groups 1, 4, 6, 9, and 11, i.e. for all the groups dosed at 1 μg. The neutralization results for each of the mRNA vaccines was improved by the presence of Matrix-M, and there was less variation across the samples administered Matrix-M.

FIG. 9B shows homologous (KP.2) pseudovirus neutralization results for Groups 2 and 7 (Pfizer: KP.2 0.5 μg and Pfizer: KP.2 0.5 μg+Matrix-M respectively). FIG. 9C shows homologous (KP.2) pseudovirus neutralization results for Groups 3, 5, 8, 10, and 12, i.e. for all the groups dosed at 0.2 μg.

FIG. 9D shows heterologous (LP.8.1) pseudovirus neutralization results for Groups 1, 4, 6, 9, and 11, i.e. for all the groups dosed at 1 μg. The neutralization results for each of the mRNA vaccines was improved by the presence of Matrix-M, and there was less variation across the samples administered Matrix-M.

FIG. 9E shows heterologous (LP.8.1) pseudovirus neutralization results for Groups 2 and 7 (Pfizer: KP.2 0.5 μg and Pfizer: KP.2 0.5 μg+Matrix-M respectively). FIG. 9F shows heterologous (LP.8.1) pseudovirus neutralization results for Groups 3, 5, 8, 10, and 12, i.e. for all the groups dosed at 0.2 μg.

Various modifications, equivalent processes, as well as numerous structures to which the present invention may be applicable will be readily apparent to those of skill in the art to which the present invention is directed upon review of the present specification. The claims are intended to cover such modifications and devices.

As noted above, the present invention is applicable to immunological compositions for VZV vaccines. Accordingly, the present invention should not be considered limited to the particular examples described above, but rather should be understood to cover all aspects of the invention as fairly set out in the attached claims.

1. An immunogenic composition, comprising a messenger ribonucleic acid (mRNA) that encodes an antigenic polypeptide, and a saponin-based matrix adjuvant.

2. The immunogenic composition of claim 1, wherein the saponin-based matrix adjuvant comprises Matrix-A and Matrix-C.

3. The immunogenic composition of claim 2, wherein the Matrix-A and Matrix-C are respectively present at a weight-to-weight ratio of about 85:15.

4. The immunogenic composition of claim 1, wherein the saponin-based matrix adjuvant is a dose of about 5 μg to about 200 μg.

5. The immunogenic composition of claim 1, further comprising a lipid nanoparticle.

6. The immunogenic composition of claim 5, wherein the lipid nanoparticle comprises a PEG-modified lipid, a non-cationic lipid, a sterol, an ionizable cationic lipid, or any combination thereof.

7. The immunogenic composition of claim 5, wherein the lipid nanoparticle comprises about 0.5-15 mol % PEG-modified lipid; about 5-25 mol % non-cationic lipid; about 25-55 mol % sterol; and about 20-60 mol % ionizable cationic lipid.

8. The immunogenic composition of claim 1, wherein the antigenic polypeptide is one of a SARS-CoV-2 spike (S) protein, MERS, SARS, and an influenza virus antigenic polypeptide.

9. The immunogenic composition of claim 1, wherein the antigenic polypeptide is a picornavirus capsid polyprotein, wherein the capsid polyprotein comprises a viral P1 precursor polyprotein.

10. The immunogenic composition of claim 1, further comprising a recombinant protein antigen.

11. The immunogenic composition of claim 10, wherein the recombinant protein antigen comprises hemagglutinin of at least one strain of seasonal influenza.

12. The immunogenic composition of claim 11, wherein the recombinant protein antigen comprises hemagglutinin of at least three strains of seasonal influenza.

13. The immunogenic composition of claim 1, wherein a dose of the composition produces an immunogenic response, measured using an IgG titer, in a murine subject that is at least twice the immunogenic response generated in a comparable murine subject exposed to the same dose of mRNA, but without the saponin-based adjuvant.

14. A method of generating an immune response in a subject, comprising administering the immunogenic composition of claim 1 to the subject.

15. The method of claim 14, further comprising combining the mRNA with the saponin-based adjuvant more than 12 hours prior to administering the composition to the subject.

16. The method of claim 14, further comprising combining the mRNA with the saponin-based adjuvant more than 24 hours prior to administering the composition to the subject.
